(19) United States
(12) Patent Application Publication
(10) Pub. No.: US 2026/0021105 A1
(43) Pub. Date: Jan. 22, 2026

(54) METHOD FOR TREATING PSORIASIS

(71) Applicant: Reviva Pharmaceuticals, Inc., Cupertino, CA (US)

(72) Inventors: Laxminarayan Bhat, Cupertino, CA (US); Seema Rani Bhat, Cupertino, CA (US)

(21) Appl. No.: 19/340,610

(22) Filed: Sep. 25, 2025

Related U.S. Application Data
(63) Continuation of application No. PCT/US2024/022075, filed on Mar. 28, 2024.
(60) Provisional application No. 63/493,241, filed on Mar. 30, 2023.

(51) Int. Cl. A61K 31/538; A61K 9/06; A61K 9/127; A61K 47/36; A61P 17/06

(52) U.S. Cl. CPC A61K 31/538; A61K 9/06; A61K 9/127; A61K 47/36; A61P 17/06

(57) ABSTRACT

The present invention is directed to a method for treating psoriasis. The method comprises administering an effective amount of brilaroxazine, or a pharmaceutically acceptable salt thereof to a subject in need thereof. Topical administration is a preferred route of administration. Brilaroxazine is effective to reduce one or more signs and/or symptoms of psoriasis.

### CROSS-REFERENCE TO RELATED APPLICATION(S)

This application is a continuation of PCT/US2024/022075, filed Mar. 28, 2024; which claims the benefit of U.S. Provisional Application No. 63/493,241, filed Mar. 30, 2023. The contents of the above-identified applications are incorporated herein by reference in their entirety.

### FIELD OF THE INVENTION

The present invention relates to methods for treating psoriasis, comprising administering to a subject in need thereof an effective amount of brilaroxazine, or a pharmaceutically acceptable salt thereof.

### BACKGROUND

Psoriasis is a systemic immune-mediated inflammatory disease with genetic components characterized by recurrent episodes of hyperkeratotic, erythematous plaques on the skin. (Kamiya 2019, Aleem 2018). With a global prevalence of ˜125 million, this condition manifests as phenotypically distinct subtypes, with plaque psoriasis accounting for >80% (Armstrong 2020, Raharja 2021). It significantly impairs the psychosocial functioning of patients, decreasing their quality of life and, in extreme cases causing depression, anxiety, or even suicidal ideation. (Marek-Josefowicz 2022). In patients with mental illness, there appears to be a higher prevalence.

Pathologically, psoriasis triggers the inflammatory skin response through extrinsic (e.g., environmental, physical, and lifestyle stressors) and intrinsic (e.g., mental and cardiometabolic stressors) risk factors (Kamiya 2019). These stressors drive the activation of innate immunocytes (e.g., dendritic cells) and the differentiation of adaptive immunocytes (e.g., T cells into Th1 cells), which subsequently release pro-inflammatory cytokines. (Cantrell 2018). These cytokines (e.g., tumor necrosis factor [TNF]-α, interferon-γ, and interleukins) lead to downstream abnormal proliferation, dysfunctional differentiation, and leucocyte infiltration of lesional keratinocytes. (Armstrong 2020). Systemic cytokine circulation increases the risk of psoriatic arthritis, cardiometabolic diseases, and physiological conditions. (Tashiro 2022, Wu 2022, Amin 2020).

Psoriasis is a long-lasting, non-contagious autoimmune disease characterized by raised areas of abnormal skin. These areas are red, pink, or purple, dry, itchy, and scaly. Psoriasis varies in severity from small, localized patches to complete body coverage.

The five main types of psoriasis are plaque, guttate, inverse, pustular, and erythrodermic. Plaque psoriasis, also known as psoriasis vulgaris, makes up about 90% of cases. It typically presents as red patches with white scales on top. Areas of the body most commonly affected are the back of the forearms, shins, navel area, and scalp. Guttate psoriasis has drop-shaped lesions. Pustular psoriasis presents as small, non-infectious, pus-filled blisters. Inverse psoriasis forms red patches in skin folds. Erythrodermic psoriasis occurs when the rash becomes very widespread and can develop from any of the other types. Fingernails and toenails are affected in most people with psoriasis at some point in time. This may include pits in the nails or changes in nail color.

Psoriasis is generally thought to be a genetic disease that is triggered by environmental factors. Symptoms often worsen during winter and with certain medications, such as beta blockers or NSAIDs. Infections and psychological stress can also play a role. The underlying mechanism involves the immune system reacting to skin cells. Diagnosis is typically based on the signs and symptoms.

Psoriasis is an autoimmune chronic-residual skin inflammatory disease, characterised by hyper proliferation of keratinocytes with erythema plaques, hyperkeratosis, and silvery-coated scales-symmetrical distribution of predilection areas of the extensor, scalp, and lumbosacral region. The exact cause is unknown, but several predisposing factors such as genetics, environmental factors, trauma, infection, drugs and psychological stress. It causes red, itchy scaly patches, most commonly on the knees, elbows, trunk and scalp.

Psoriasis signs and symptoms can vary from person to person. Common signs and symptoms include:
[symbol] Red patches of skin covered with thick, silvery scales
[symbol] Small scaling spots (commonly seen in children)
[symbol] Dry, cracked skin that may bleed or itch
[symbol] Itching, burning or soreness
[symbol] Thickened, pitted or ridged nails
[symbol] Swollen and stiff joints

Very rapid multiplication of keratinocytes occurs in people having psoriasis, and their movement from the stratum basale (basal layer) to the upper layer of epidermis occurs in 4 days. Thick dry patches or plaques form, as the skin does not shed the cells quickly. Very mild psoriasis exists in some people, which cannot even be suspected as a skin disorder. Very severe psoriasis may be seen in others, sometimes in which the whole body is covered with scaly, thick, red skin. Despite the fact that psoriasis occurs in a population of all age groups, i.e., pediatrics to geriatrics, generally, it is diagnosed in the adolescence of a person. The other causative factors for psoriasis are genetics, sudden changes in genes (mutations), climate, immune system abnormality, mental or emotional strain, contagion, and wounds.

Current treatments of psoriasis include topical therapy, phototherapy, and systemic therapy, with the latter reserved for severe cases. Topical corticosteroids and Vitamin D derivatives remain first-line as monotherapy and complementary to systemic therapy, despite the drawbacks of limited systemic efficacy and long-term side effects (e.g., tachyphylaxis, skin atrophy, adrenal suppression, and skin irritation). Topical treatments can rapidly act and exert localized effects with minimal short-term adverse events. Conventional nonbiologic oral agents (e.g., methotrexate, apremilast, acitretin, or cyclosporine) offer more options for treating widespread inflammation; however, they are associated with significant toxicities (e.g., hepatotoxicity, nephrotoxicity, hypertension, dyslipidemia, malignancy, and teratogenicity). (Armstrong 2020, Jain 2021).

Biologics (e.g., TNF and IL inhibitors) target specific immune response components. However, these agents' use is limited due to the potential development of immunogenicity, serious infection and malignancy risk, parenteral administration, and affordability. Undertreatment remains a concern, especially in severe cases and special populations, considering the complexity of managing a multisystem disease. (Raimondo 2017, Feldman 2016). Hence, there is a need for new effective treatments with acceptable safety profiles and convenient administration routes that allow for a more personalized treatment approach. (Rendon 2019, Jiang 2023).

Brilaroxazine (RP5063) is a multimodal dopamine and 5-HT receptor modulator. Brilaroxazine displays a high binding affinity to D2-4 and 5-HT1A receptors as partial agonists, 5-HT2A as a weak partial agonist or neutral antagonist, 5-HT2B/7 as an antagonist, and a moderate affinity to serotonin transporter (SERT). Brilaroxazine has an established efficacy, safety, and pharmacokinetic profile from phase 1 and 2 studies in healthy volunteers and schizophrenia patients. Also, preclinical work indicates that this agent inhibits the release of multiple proinflammatory cytokines.

Liposomes are micro-particulate or colloidal carrier systems, usually 0.025-5.0 μm in diameter. Liposomes are composed of biodegradable, biocompatible components and provides a unique opportunity to deliver pharmaceuticals into the cells or even inside individual cellular compartments. Liposomes form spontaneously when the lipids are hydrated in aqueous media at the transition temperature. Lipids are composed of natural and/or synthetic lipids (phospholipids and sphingolipids) and may contain other bilayer constituents such as cholesterol and hydrophilic polymer lipids. FIG. 1 shows a representation a general structure of liposomes.

The compositions of liposomes determine interaction with blood and tissues. The compositions decide liposome's net physicochemical properties, namely membrane fluidity, charge density, and steric hindrance permeability. They are found to be useful carriers for both hydrophilic and hydrophobic drugs. These drug delivery systems employed for the delivery of drugs with varying lipophilicities, like water-soluble drug will be encapsulated within the aqueous compartment; the lipophilic drug is usually bound to the lipid bi-layer or dissolved in the lipid phase.

### BRIEF DESCRIPTION OF THE DRAWINGS

FIG. 1 shows a diagram of a general structure of a liposome.

FIG. 2 shows particles size (Z-average) and particles distribution index in the liposomal dispersion by DLS measurement of brilaroxazine liposomes.

FIG. 3 shows the HPLC chromatogram of brilaroxazine liposomes.

FIG. 4 shows the HPLC chromatogram of a lipogel sample. The first peak is brilaroxazine and the second peak is excipient.

FIG. 5 shows in-vitro diffusion study graph of lipogel through the membrane.

FIG. 6 shows the comparative effects on Psoriasis Area and Severity Index (PASI) from Days 1 to 12 in the imiquimod-induced psoriatic mouse model. PASI, between brilaroxazine lipogel and induced Psoriasis group from Days 3 through 12 (p=0.03).

FIG. 7 shows the Baker scores for Sham control, psoriasis, and brilaroxazine lipogel groups in the imiquimod-induced psoriatic mouse model.

FIG. 8 shows pictures of 100× magnification of skin histology studies by H&E staining.

FIG. 9 shows pictures of 400× magnification of skin histology studies by H&E staining.

FIG. 10 shows serum TNF alpha level in different study group animals.

FIG. 11 shows serum KI67-level in different study group animals.

FIG. 12 shows serum TGF-beta level in different study group animals.

### DETAILED DESCRIPTION OF THE INVENTION

The present invention is directed to a method of treating psoriasis by administering an effective amount of brilaroxazine to a subject in need thereof. Brilaroxazine is effective to reduce one or more signs or symptoms of psoriasis.

Brilaroxazine is a dopamine-serotonin system stabilizer with potent partial agonist activity at the dopamine D2, D3, and D4, and serotonin 5-HT1A and 5-HT2A receptors, and antagonist activity at the serotonin 5-HT6 and 5-HT7 receptors. By having potent partial agonist activity at serotonin 5-HT1A and 5-HT2A receptors, brilaroxazine reduces the production of inflammatory mediators such as TNF-α, IFN-γ, IL-1β, IL-6, IL-8, and prevents the activation on nuclear factor-KB to induce activation of keratinocytes, trigger deterioration of keratinocytes, and worsen psoriasis symptoms.

Brilaroxazine (free based) is a basic and lipophilic molecule and has a molecular weight of 450.36 g/mol. Its chemical structure is shown below.

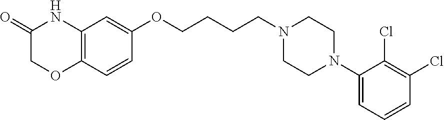 [SMILES: O=C1COc2ccc(OCCCCN3CCN(c4cccc(Cl)c4Cl)CC3)cc2N1]

Brilaroxazine often is the HCl salt form with a molecular weight of 486.7 g/mol.

### Pharmaceutical Compositions

The present invention provides pharmaceutical compositions comprising one or more pharmaceutically acceptable carriers and brilaroxazine, or a pharmaceutically acceptable salt thereof. Brilaroxazine or its pharmaceutically acceptable salt in the pharmaceutical compositions in general is in an amount of about 0.01-20%, or 0.05-20%, or 0.1-20%, or 0.1-10%, or 0.1-5%, or 0.1-2%, or 0.2-15%, or 0.2-10%, or 0.2-5%, or 0.2-2%, or 1-5% (w/w) for a topical formulation; about 0.1-5% for an injectable formulation, 0.1-5% for a patch formulation, about 1-90% for a tablet formulation, and 1-100% for a capsule formulation.

In one embodiment, brilaroxazine is incorporated into any acceptable carrier, including creams, gels, lotions or other types of suspensions that can stabilize the active compound and deliver it to the affected area by topical applications. In another embodiment, the pharmaceutical composition can be in a dosage form such as tablets, capsules, granules, fine granules, powders, syrups, suppositories, injectable solutions, patches, or the like. The above pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Other pharmaceutically acceptable carriers include xanthan gum, carrageenan, Avicel RC-591 (a combination of microcrystalline cellulose and), and polyethylene glycol. Alternately, the active compound may be dissolved or suspended in a pharmaceutically acceptable lipid formulation such as those described by Kalepu et al (Acta Pharmaceutica Sinica B, 3:361-372, 2013), for example, vegetable oil, coconut oil, castor oil, etc.

Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the brilaroxazine may contain other excipients that have no bioactivity and no reaction with the active compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of excipients of a tablet or a capsule include, but not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone. For example, a tablet formulation may contain inactive ingredients such as colloidal silicon dioxide, crospovidone, hypromellose, magnesium stearate, microcrystalline cellulose, polyethylene glycol, sodium starch glycolate, and/or titanium dioxide. A capsule formulation may contain inactive ingredients such as gelatin, magnesium stearate, and/or titanium dioxide.

For example, a patch formulation of brilaroxazine may comprise some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters or diethylene glycol monoethyl ether.

Topical formulations including brilaroxazine can be in a form of gel, cream, lotion, liquid, emulsion, ointment, spray, solution, and suspension. The inactive ingredients in the topical formulations for example include, but not limited to, (emollient/permeation enhancer), diethylene glycol monoethyl ether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent).

The present application further provides a gel formulation comprising brilaroxazine liposome, a gelling agent and a humectant. In one embodiment, the gel formulation comprises brilaroxazine in an amount of 0.005-10%, 0.01 to 5%, or 0.1-2% by weight. In one embodiment, the formulation has a gel appearance, and the bilayer lipid vesicles are intact and stable in the gel. In one embodiment, the gelling agent is carbomer 940, and the humectant is glycerin.

### Brilaroxazine Liposomes

In one embodiment, brilaroxazine is incorporated in bilayer lipid vesicles of a liposome composition. In one embodiment, the liposome composition comprises bilayer lipid vesicles encapsulating an aqueous solution, wherein the bilayer lipid vesicles comprise one or more phospholipids, sterol, and brilaroxazine.

The lipids used in forming the lipid vesicles typically include lipid mixtures composed predominantly of phospholipid(s) and sterol(s). A list of phospholipids used commonly in liposome preparations can be found on page 471 of Szoka et al (Ann Rev Biophys Bioeng (1980) 9:467). The vesicles may be formulated to include negatively or positively charged lipids, such as phosphatidic acid (PA) and phosphatidylglycerol (PG), to provide a desired surface charge on the reagent vesicles. A small amount of antioxidant such as α-tocopherol (0.1 to 1 mol %) can be added to the lipid mixture to increase the stability. A typical lipid mixture used in forming brilaroxazine liposome of the present invention includes phosphatidylcholine, cholesterol, and brilaroxazine.

In one embodiment, the aqueous solution of the liposome composition comprises maltodextrin. Maltodextrin consists of D-glucose units connected in chains of variable length. The glucose units are primarily linked with α(1→4) glycosidic bonds. Maltodextrin is typically composed of a mixture of chains that vary from three to 17 glucose units long. Brilaroxazine liposomes encapsulating maltodextrin may provide a better drug release profile than that of brilaroxazine liposomes without maltodextrin.

The brilaroxazine liposomes are prepared by first dissolving the vesicle-forming lipids (e.g., brilaroxazine, phosphatidylcholine, cholesterol) in an inert organic solvent or solvent system, for example, chloroform and/or ethanol, to form an organic-phase solution of the lipids. In general, the inert organic solvent or solvent system is one in which the lipid components can be readily dissolved, at the concentration in the range of between about 0.5-50 mg lipid/mL. Then the lipids solution is dried completely to remove the organic solvent(s) and forms a thin lipid film on the surface of a vessel. After drying, the thin lipid film is then hydrated with an aqueous solution. In a preferred embodiment, the aqueous solution contains maltodextrin.

In one embodiment, the brilaroxazine liposomes comprise 10-40% or 20-30% by weight of brilaroxazine.

In one embodiment, the brilaroxazine liposomes comprises 20-60% or 30-45% by weight of maltodextrin.

In one embodiment, the average particle size of the brilaroxazine liposomes in a formulation is between 500-750 nm.

In one embodiment, the most intense peak of the brilaroxazine liposomes in a formulation has a peak size between 900-1000 nM.

### Method for Treating Psoriasis

The present application provides a method for treating psoriasis. The method comprises administering an effective amount of brilaroxazine to a subject in need thereof. “An effective amount,” as used herein, is the amount effective to treat psoriasis by ameliorating the pathological condition or reducing the symptoms of psoriasis. The method reduces the one or more signs and symptoms selected from the group consisting of: red patches of skin covered with thick and silvery scales; small scaling spots; dry and cracked skin; itching, burning, or soreness of the skin; thickened, pitted, or ridged nails, and swollen and stiff joints.

The pharmaceutical composition of the present invention can be applied by local administration and systemic administration. Local administration includes topical administration. Topical administration is a preferred route of administration.

In topical administration, brilaroxazine may be contained in a topical dosage form and contact directly on the psoriatic plaques. Topical delivery is applying the brilaroxazine formulation to the skin to directly treat the cutaneous disorder or the cutaneous manifestations of a disease with the intent of containing the pharmacological or the effect of brilaroxazine to the surface of the skin. Topical formulations are applied to minimize the flux of brilaroxazine through the skin and maximizing its retention on the skin. The therapeutic effect of topical formulations depends on the ability of brilaroxazine to penetrate the skin layers which, in turn, depends on the physicochemical properties of brilaroxazine, the carrier base, and the skin conditions. Topical formulations for the treatment of psoriasis can be administered in a wide variety of pharmaceutical forms: ointments, creams, gels, lotions, sprays, foams, etc. Through a topical delivery, brilaroxazine provides its serotonin mechanism in the cells to control psoriasis. As a topical dosage form, less dosage strength is sufficient than the oral dosage form to provide the required pharmacodynamics action at the site of application.

Commonly used conventional semisolid dosage forms have certain limitations in drug delivery due to barrier properties of the skin. The skin is continuously involved in the construction of an efficient homeostatic barrier. Liposomes are a drug delivery system that can be used for topical delivery of drug molecules. Liposomes are microscopic vesicles that contain amphipathic phospholipids arranged in one or more concentric bilayers enclosing an equal number of aqueous compartments. In this form, as a spherical shell, liposomes resemble biological membranes. Liposomes comprise biodegradable, biocompatible components and provides a unique opportunity to deliver pharmaceuticals into the cells or even inside individual cellular compartments. Hence, brilaroxazine liposomes dispersed semisolid dosage forms may offer significant advances to deliver brilaroxazine in the deeper layer of the skin in severe psoriatic conditions.

Systemic administration includes oral, parenteral (such as intravenous, intramuscular, subcutaneous or rectal), and other systemic routes of administration. In systemic administration, the active compound first reaches plasma and then distributes into target tissues.

In one embodiment, the composition is applied topically onto the affected area and rubbed into it. The composition is topically applied at least 1 or 2 times a day, or 3 to 4 times per day, depending on the medical issue and the disease pathology being chronic or acute. In general, the topical composition comprises about 0.01-10% (w/w) of the active compound brilaroxazine. For example, the topical composition comprises about 0.1 to 2% (w/w) of the active compound. Depending on the size of the affected area, 0.2-85 mL, typically 0.2-10 mL, of the topical composition is applied to the individual per dose. The active compound passes through skin and is delivered to the site of discomfort.

Those of skill in the art will recognize that a wide variety of delivery mechanisms are also suitable for the present invention.

The present brilaroxazine liposomes composition is useful in treating a mammal subject, such as humans, horses, and dogs. The present invention is particularly useful in treating humans.

The following examples further illustrate the present invention. These examples are intended merely to be illustrative of the present invention and are not to be construed as being limiting.

### Examples

### Example 1. Preparation of Brilaroxazine Liposomes

Table 1 shows the formulation composition of brilaroxazine liposomes.

TABLE 1

| No. | Ingredients list | Percentage | CAS No. |
|---|---|---|---|
| 1. | Brilaroxazine | 24.53 | 1239729-06-6 |
| 2. | Lecithin | 34.76 | 8002-43-5 |
| 3. | Cholesterol | 2.97 | 57-88-5 |
| 4. | Maltodextrin | 37.74 | 9050-36-6 |
| 5. | Purified water (for | Qs | NA |
| | lipid film hydration) | | |

Solvents used for lipid dry film preparation

| | | | |
|---|---|---|---|
| 6. | Chloroform (dried | NA | 67-66-3 |
| | out in the process) | | |
| 7. | Ethanol (dried out | NA | 64-17-5 |
| | in the process) | | |

The brilaroxazine liposomes were prepared by the lipid hydration method. Briefly, phosphatidylcholine and cholesterol were dissolved in a suitable solvent (chloroform and/or ethanol) and brilaroxazine was dissolved in the same solvent. Then this drug-lipid solution was dried at 45-50° C. in a rotary evaporator using the vacuum to remove the solvent completely. Once all the solvent was removed, a thin film formed in the round bottom flask. This round bottom flask containing lipid film was kept in a vacuum for 12-24 h to completely remove the traces of the solvents present in the thin lipid film. After 12-24 h of drying, the thin film was hydrated with 66 mL of 60° C. maltodextrin solution (concentration 39.57 mg/mL).

### Example 2. Particles Size and Zeta Potential Analysis of Brilaroxazine Liposomes

The prepared liposomes from Example 1 were viewed at different magnifications in an optical microscope for confirmation during the hydration process. Optical microscopic viewing at different magnifications (10×, 20×, and 40×) for confirmation of the prepared spherical-shaped liposomal vesicles occurred throughout the hydration process.

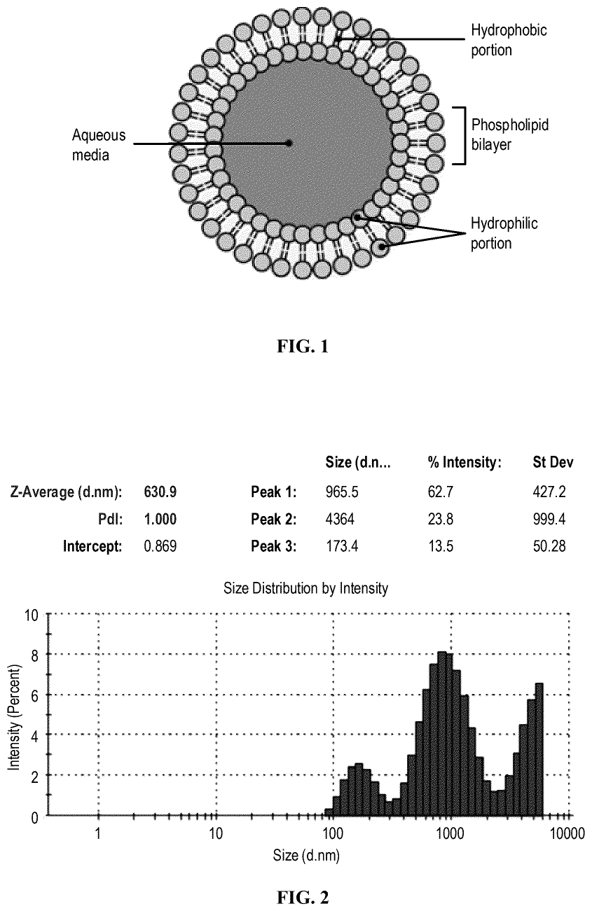

The liposomes were analyzed for particle size by DLS (dynamic light scattering) method for particle size analysis and drug content. The Z-average (particle size) was measured for the prepared liposomes. FIG. 2 shows particles size (Z-average) and particles distribution index in the liposomal dispersion by DLS measurement of brilaroxazine liposomes.

Zeta potentials were estimated from experimentally determined electrophoretic mobility of particles. The value of zeta potential indicates the stability of the colloidal dispersion.

Zeta potential (mV) value:
[symbol] 0 to 5—Rapid coagulation or flocculation
[symbol] 10 to 30—Incipient instability
[symbol] 30 to 40—Moderate stability
[symbol] 40 to 60—Good stability
[symbol] >61—Excellent stability

In general, the colloidal dispersion with zeta potential values greater than positive 30 mV or less than negative 30 mV have high degrees of stability. The higher the zeta potential value (both positive and negative), the better the stability.

Table 2 shows particle size and zeta potential analysis of Liposomes by DLS method.

TABLE 2

| | Particle Size | Zeta |
|---|---|---|
| | (Z-Average) | potential |
| Liposomes No. | nm | (mV) |
| RP5063 Liposomes | 630.9 | 38.8 |

### Example 3. Measurement of Drug Content by HPLC

Liposome drug content was analyzed by the HPLC method.

### HPLC System

Shimadzu HPLC system (LC-2030C Plus, Serial No: L21445711704 AE, Made in Japan), auto sampler, UV detector, data acquisition system. An equivalent system may be used as a substitute.

### HPLC Column

Shimadzu Shim-Pack GIST C18, 5 μm, 250×4.6 mm column or equivalent.

### Reagent Preparation

### Mobile Phase A

[symbol] Dissolve 2.72 g KH2PO4 in 1000 mL of ultrapure water (0.02M Solution).
[symbol] Adjust the pH to 3.0 with phosphoric acid.
[symbol] Mix 90 parts of the above buffer with 10 parts of acetonitrile.
[symbol] Filter through the membrane prior to use.

### Mobile Phase B

[symbol] Mix 90 parts of acetonitrile with 10 parts of ultrapure water. Adjust the pH to 3.0 with phosphoric acid.
[symbol] Filter through the membrane prior to use.

### Diluent

Prepare a mixture of acetonitrile and phosphate buffer (section 6.2.1.1.) (85:15) for drug content/encapsulation efficiency.

### Sample Preparation for Drug Content and Incarnation Efficiency

### Drug Content

Take the required amount of liposomes sample (whole dispersion) in a volumetric flask, add the volume of diluent needed (6.2.3), and mix well. Keep this mixture in a bath sonicator for 30-45 minutes at 60° C. Take the required volume of the prepared sample and dilute it to the required concentration. The assay/drug content concentration is 20 μg/mL.

### Incorporation Efficiency

Take the required amount of liposomes sample in a centrifuge tube and centrifuge the liposomes dispersion at 10000 rpm for 30 minutes at 20° C. Remove the supernatant solution and collect the pellet. Add the required diluent volume to the liposomal pellet, and mix well. Keep this mixture in a bath sonicator for 30-45 minutes at 60° C. Take the required volume of the prepared sample and dilute it to drug concentration of 20 μg/mL.

### Standard Solution

Prepare 20 μg/mL standard solution with the above diluent.

### Analysis

Set up the HPLC using the following parameter:

Column: Shimadzu Shim-Pack GIST C18, 5 μm, 250×4.6 mm column or equivalent

Flow rate: 1 mL/min

Injection volume: 20 μL

Detection: UV @215 nm

Column Temperature: 30° C.

Run time: 5-7 minutes

HPLC conditions: Mobile phase A, 25%; mobile phase B, 75%

### Identification

Compare the retention time of the standard/pure drug peak and sample drug peak

### Result

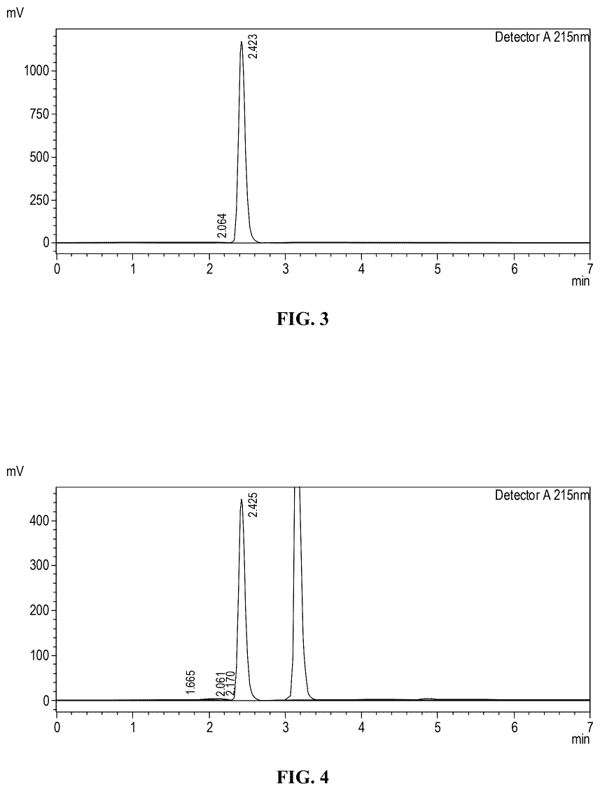

The HPLC chromatogram is shown in FIG. 3. The HPLC results show that drug content is 96% and drug incorporation efficiency into liposome is 73%.

The final formulation composition was consistent with brilaroxazine (24.53%), lecithin (34.75%), cholesterol (2.97%), maltodextrin (37.74%), and purified water for lipid film hydration.

### Example 4. Preparation of Liposomal Gel Formulations

The liposomal gel was prepared by incorporating a liposome dispersion in a gel formulation. First, the plain gel was prepared, and then the liposome dispersion was added and mixed thoroughly to result in the liposomal gel or lipogel. Various percentages of lipogel formulation (0.25% to 1.5% of brilaroxazine) were prepared according to need. Table 3 shows the composition of the lipogel formulation.

TABLE 3

| | Lipogel | |
|---|---|---|
| Ingredients | Formulation - | |
| activity | RPLG | Percentage (%) |
| Gelling agent | Carbomer 940 | 0.65-0.85 |
| Humectant | Glycerin | 5.00 |
| pH modifier | Triethanolamine | 0.10-0.25 |
| Preservative | Phenoxyethanol | 1.00 |
| Vehicle | Purified water | Qs |
| Brilaroxazine | RP5063 liposomal | Eq to 0.25% or |
| | dispersion | 1.5% of brilaroxazine |

The prepared liposomal gel formulation was evaluated for physical appearance and pH. All the gel formulations were viewed under an optical microscope for intact liposomes in the gel formulation. The liposomal gel formulation had white cream gel appearance, with pH 5-6, and microscope examination showed presence of liposomes. In addition, the liposome particles were intact in all gel formulations and stable.

### Example 5. Analysis of Lipogel by HPLC

Lipogel sample was placed in a volumetric flask and added with diluent and mixed well. Brilaroxazine content was analyzed by HPLC according to the same protocols of Example 3.

Comparing the retention time of the pure drug peak and sample peak, the drug content is calculated to be 95.12%.

The HPLC chromatogram of a lipogel sample is shown in FIG. 4. The first peak is brilaroxazine and the second peak is excipient. Brilaroxazine peak is clear and separated from the excipient peak.

### Example 6. In Vitro Diffusion/Permeation Studies of Lipogel

The prepared lipogels were analyzed for drug diffusion/permeation using a Franz diffusion cell. The Franz diffusion cell was filled with pH 7.4 PBS buffer. The surface treated and pH 7.4 PBS neutralized regenerated cellulose dialysis membrane (MW cut off: 12000 to 14000) was placed on the receptor compartment, and a weighed amount of lipogel was placed in the donor compartment. The receptor solution was stirred with a magnetic stirrer, and the skin temperature was maintained in the diffusion cell by the circulation of temperature-controlled water in the outer jacket. At different time intervals, samples were withdrawn through a sample port and replaced with the same volume of plain PBS. The withdrawn sample was mixed with an equal volume of HPLC diluent and analyzed for drug content at different time intervals. The percentage of drug diffusion, flux rate, and permeation coefficient was calculated for each lipogel formulation. The time vs percentage drug diffusion/release was plotted using Graph Pad Prism Version 6.01 Software.

### HPLC Analysis of Drug Diffusion Samples

The HPLC analysis method, system, and columns were the same as described in Example 3 except a mixture of acetonitrile and phosphate buffer (60:40) was used for drug diffusion/permeation studies.

### Sample Preparation for Diffusion/Permeation Sample Analysis

Mix an equal volume of diluent with diffusion/permeation sample fluid taken from the Franz diffusion apparatus. Mix thoroughly in a vertex mixer and filter the solution through a syringe filter.

### Results

The results of HPLC chromatogram of the lipogel diffusion sample show a clear and separated brilaroxazine peak.

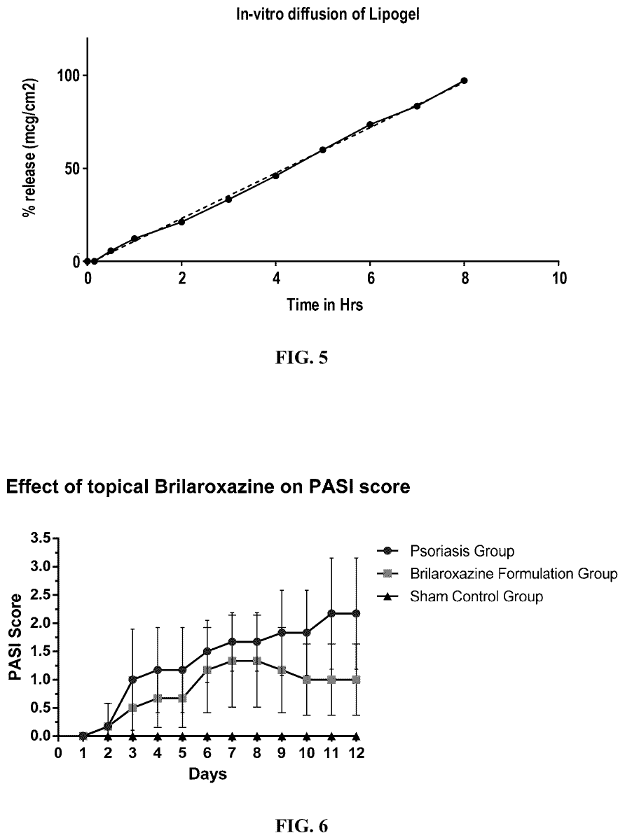

FIG. 5 shows in-vitro diffusion study graph of lipogel through the membrane. The release profile showed steady and sustained brilaroxazine release from the formulation throughout the study period of 8 hours.

Table 4 shows in vitro diffusion study results of Lipogel formulations.

TABLE 4

| | Flux | Permeation | |
|---|---|---|---|
| Formulation | (μg/cm²/h) | coefficient (Cm/h) | R2 Value |
| Lipogel | 12.02 | 3.28 | 0.9984 |
| Formulation | | | |
| With maltodextrin | | | |
| Lipogel | 5.48 | 1.92 | 0.9633 |
| Formulation | | | |
| Without | | | |
| maltodextrin | | | |

The lipogel formulation containing liposomes with maltodextrin show a better drug release profile and the higher flux and permeation value in the in vitro diffusion study than that of liposomes without maltodextrin. This may be due to the increased solubility of RP5063 in the liposomal gel and optimum particle size distribution in the formulation.

### Example 7. In Vivo Preclinical Studies—Psoriasis Model

BALB/c mice (n=6/group) were used for this experiment, and animals were maintained as per Institutional Animal Ethics Committee-approved protocol. Imiquimod cream (5%) was used as an induction chemical for Psoriasis disease pathology. Psoriasis was induced by applying Imiquimod on the back side of the animal's shaved skin in the morning for 12 days. The test lipogel formulation was used on the animals in the evening hours for 12 days. Imiquimod was applied till the last day of the experiment. All animals were observed for the following parameters: PASI score, Baker's score, histology H&E staining, and serum cytokine analysis (TNF-alpha, KI67, TGF-beta). Table 5 shows animal groups used for preclinical studies.

TABLE 5

| | | No. of | Treatment |
|---|---|---|---|
| Group | Formulations used | mice | days |
| Group 1 | Normal control group | 6 | — |
| Group 2 | Psoriasis control group | 6 | 1-12 |
| Group 3 | Psoriasis + Lipogel | 6 | 1-12 |
| | formulation group | | |

### PASI Score

The animals were observed for the sign of imiquimod-induced psoriasis toxicity each day, and the PASI score was calculated for each group. FIG. 6 illustrates the composite PASI score for Days 1-12. The induced group (Psoriasis group) displayed higher PASI scores than the non-induced control group (Sham control group) (p=0.001). The differences in magnitude grow larger between these two groups starting from Day 3 out to Day 11. The brilaroxazine Lipogel group increased PASI scores starting on Day 3, peaking at Days 7 and 8, and fell to a plateau level on Days 10 through 12. The scores were higher in magnitude than the Sham control group but did not reach the same level as the Psoriasis group. Brilaroxazine Lipogel PASI scores were consistently lower than those in the induced Psoriasis group from Days 3 through 12 (p=0.03). The maximum difference in magnitude appeared son Days 11 and 12.

### Baker's Score

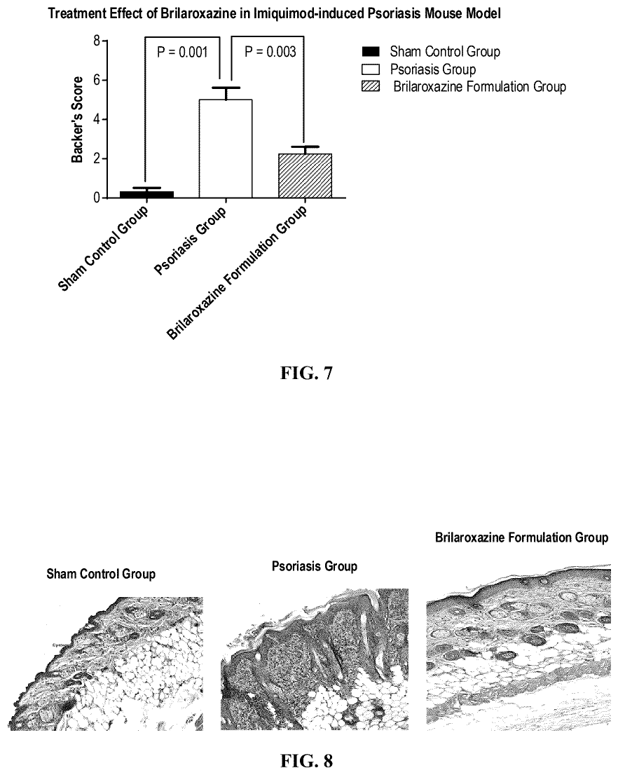

At the end of the 12th day of the study period, the animals were sacrificed, the skin was collected, and histology was performed. The sign of psoriasis toxicity was checked on each animal's skin, and Baker's score was calculated for each group. Baker's score showed a significant reduction in the lipogel formulation-treated group animals. FIG. 7 shows that topical brilaroxazine formulation significantly (P=0.003) treated the psoriasis animals.

### Histology (H&E Staining)

FIG. 8 shows pictures of 100× magnification of skin histology studies. Table 6 describes observation of skin histology samples at 100× magnification.

TABLE 6

| Group | Observations (100x) |
|---|---|
| Sham control | Normal epidermis (1-3 layers), no inflammatory |
| group | infiltration |
| Psoriasis | Increased epithelial layers 3-7 layers (green), |
| control group | increased keratinization, Munro's abscess |
| | (red arrow), severe inflammatory (blue) |
| | infiltration |
| Psoriasis + | Reduced epithelial thickness (3-4 layers), |
| Brilaroxazine | reduced inflammatory infiltration, absence of |
| Lipogel | parakeratinization, absence of munro's abscess |

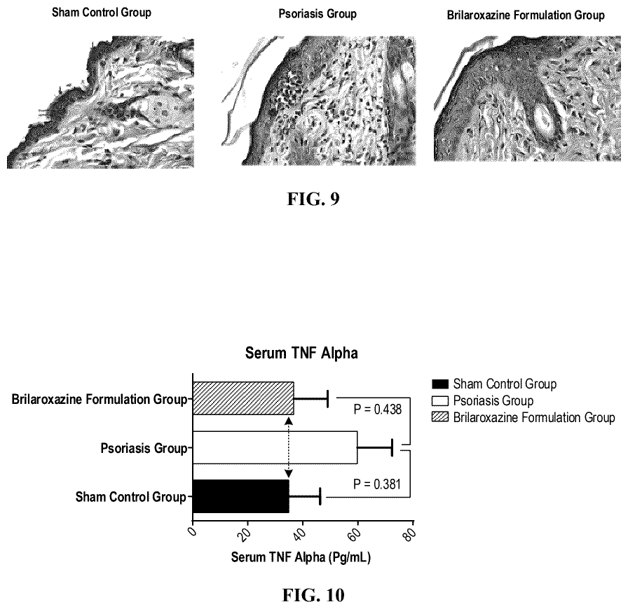

FIG. 9 shows pictures of 400× magnification of skin histology studies. Table 7 shows observation of skin histology samples at 400× magnification.

TABLE 7

| Group | Observations (400x) |
|---|---|
| Sham control | Epithelium 1-3 layers, no inflammatory infiltration, |
| group | very little keratin in stratum corneum |
| Psoriasis | Severe acute and chronic inflammatory infiltration, |
| control group | Kogoj pustule |
| Psoriasis + | Reduced epithelial thickness, reduced inflammatory |
| Brilaroxazine | infiltration |
| Lipogel | |

In summary, histology evaluation included direct observation at 100× and 400× magnification. Tables 6 and 7 and FIGS. 8 and 9 provide histological observations and H&E staining at both magnifications. Differences appeared between the Sham control with the induced Psoriasis and brilaroxazine Lipogel groups and between the latter two groups. Baker score comparisons (FIG. 7) reflected significant effects for the Sham control (p=0.001) and the Brilaroxazine Lipogel (p=0.003) groups, versus the Psoriasis cohort. Such observation highlights the treatment effect of Brilaroxazine Lipogel.

### Serum Cytokine Level

At the end of the 12th day study period, the animals were sacrificed, blood was collected, and serum was separated. The serum cytokines TNF-alpha, KI 67 and TGF-beta were analyzed by the ELISA method.

FIG. 10 shows serum TNF alpha level in different study group animals. The test results show that the brilaroxazine formulation reduced the serum TNF-alpha level in psoriasis-induced animals, and the TNF alpha level was comparable to that of the sham control group animals.

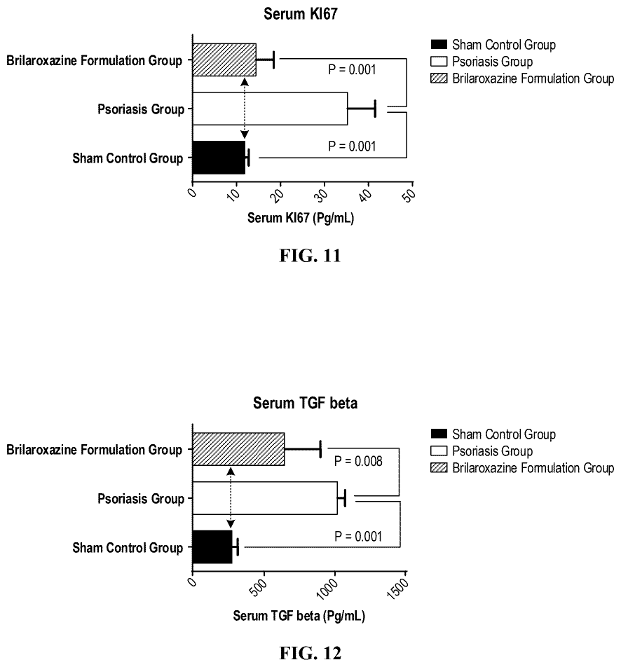

FIG. 11 shows serum KI67 level in different study group animals. The test results show that the brilaroxazine formulation significantly reduced (P=0.001) the serum KI67 level in psoriasis-induced animals, and the serum KI67 level was comparable to that of the sham control group animals.

FIG. 12 shows serum TGF-beta level in different study group animals. The test results show that the lipogel formulation significantly reduced (P=0.008) the serum TGF-beta level in psoriasis-induced animals compared to the psoriasis control group animals.

It is to be understood that the foregoing describes preferred embodiments of the present invention and that modifications may be made therein without departing from the scope of the present invention as set forth in the claims.

What is claimed is:

1. A method for treating psoriasis, comprising administering an effective amount of brilaroxazine, or a pharmaceutically acceptable salt thereof to a subject in need thereof.

2. The method of claim 1, wherein brilaroxazine is administered topically to the subject.

3. The method of claim 2, wherein brilaroxazine is incorporated in bilayer lipid vesicles of liposomes.

4. The method of claim 3, wherein the liposomes comprise one or more phospholipids, sterol, and brilaroxazine or a pharmaceutically acceptable salt thereof, and encapsulate an aqueous solution comprises maltodextrin.

5. The method of claim 4, wherein the liposomes comprise 20-30% by weight of brilaroxazine.

6. The method of claim 4, wherein the liposomes are contained in a gel formulation.

7. The method of claim 4, wherein the gel formulation comprises 0.1-2% of brilaroxazine.

8. The method of claim 1, wherein brilaroxazine is administered systemically to the subject.

9. The method of claim 1, wherein the method reduces the one or more signs and symptoms selected from the group consisting of: red patches of skin covered with thick and silvery scales; small scaling spots; dry and cracked skin; itching, burning, or soreness of the skin; thickened, pitted, or ridged nails, and swollen and stiff joints.
